(19) United States
(12) Patent Application Publication
(10) Pub. No.: US 2012/0245120 A1
(43) Pub. Date: Sep. 27, 2012

(54) ZINC SUCROSE OCTASULFATES, THEIR PREPARATION, AND PHARMACEUTICAL AND COSMETIC USES THEREOF

(75) Inventors: Pierre Fabre, Castres (FR); Séverine Jeulin, L'Hay Les Roses (FR); Marion Kopec, Toulouse (FR)

(73) Assignee: PIERRE FABRE DERMO-COSMETIQUE, Boulogne-billancourt (FR)

(21) Appl. No.: 13/514,121

(22) PCT Filed: Dec. 3, 2010
(86) PCT No.: PCT/EP10/68873

§ 371 Date: Jun. 6, 2012

(30) Foreign Application Priority Data
Dec. 7, 2009 (FR) 0958689

(51) Int. Cl. A61K 31/7135; C07H 1/00; A61K 8/60; A61Q 19/00; A61P 17/02; A61P 1/04; A61P 1/00; A61P 17/10; C07H 11/00; A61P 17/00

(52) U.S. Cl. 514/53; 536/121

(57) ABSTRACT

The present invention relates to a zinc sucrose octasulfate of the general formula I, a process for preparing same and its use in the pharmaceutical and/or cosmetic field

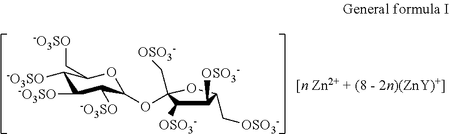

wherein:
0≦n≦4
n is an integer
Y represents OH, Cl, Br, I, NO₃, C₆H₅O₇, CH₃CO₂, CF₃CO₂, or —OCH₃.

The present invention relates to a zinc sucrose octasulfate, a process for preparing it and its use in the pharmaceutical and/or cosmetic field.

Oligosaccharides are carbohydrates which upon hydrolysis generate only oses. They are sugars consisting of at least two molecules of simple sugars (or oses) linked together. Oligosaccharides include sucrose, a double sugar formed by condensation of 2 oses: one glucose molecule and one fructose molecule.

Sulfated oligosaccharides are known from literature and have a variety of biological, cosmetic and/or therapeutic activities.

WO2006/017752 discloses a method for treating inflammations of airways using oligosaccharides as an active ingredient. Oligosaccharides further include the fully sulfated oligosaccharide obtained by condensation of glucose and condensation of fructose.

Sulfated oligosaccharides, mainly aluminum sucrose octasulfate, are also used in the treatment of alopecia (U.S. Pat. No. 5,767,104).

Sucrose octasulfate is used as an active principle in the treatment of stomach ulcers for its repairing/healing properties. FR 2 646 604 discloses formulations of aluminum sucrose octasulfate, or sucralfate, having anti-inflammatory and healing properties suitable for the treatment of wounds or other ulcerative inflammations. WO 94/00476 discloses a method for treating damages and/or inflammations of the digestive system by administration of a sulfated sucrose salt, more particularly potassium or sodium sucrose octasulfate.

FR 1 390 007 discloses the topical use of a formulation comprising sucralfate combined with copper and zinc sulfate as a tissue regenerating, healing, and soothing agent.

EP 0 230 023 discloses the use of polysulfated oligosaccharides, more particularly potassium sucrose octasulfate, as an agent for healing wounds.

The object of the present invention is to provide a new compound combining repairing, antimicrobial and antiradical properties. Such compound is found useful in the preparation of pharmaceutical and/or cosmetic compositions suitable for skin repair, for healing wounds and for enhancing cicatrization. This type of composition combines both skin treatment and antimicrobial protection.

The present invention relates to compounds of the general formula I

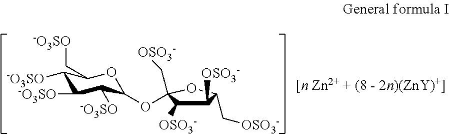

wherein:

0≦n≦4

n is an integer

Y represents OH, Cl, Br, I, NO₃, C₆H₅O₇, CH₃CO₂, CF₃CO₂, or —OCH₃.

In one embodiment of the invention, the compound is a compound of the formula II

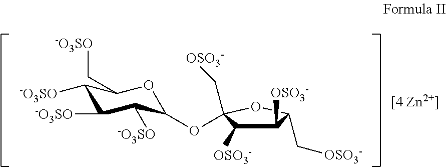

In this embodiment of the invention, the compound of the formula II is a compound of the general formula I wherein n is 4.

In one embodiment of the invention, the compound is a compound of the formula III

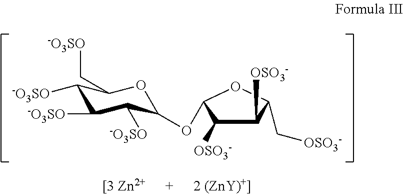

In this embodiment of the invention, the compound of the formula II is a compound of the general formula I wherein n is 3.

In one embodiment of the invention, the compound is a compound of the formula IV

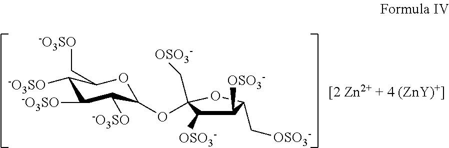

In this embodiment of the invention, the compound of the formula II is a compound of the general formula I wherein n is 2.

In one embodiment of the invention, the compound is a compound of the formula V

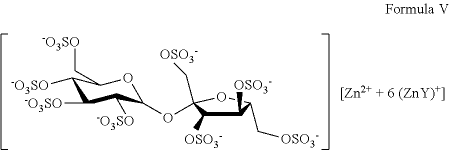

In this embodiment of the invention, the compound of the formula II is a compound of the general formula I wherein n is 1.

In one embodiment of the invention, the compound is a compound of the formula VI

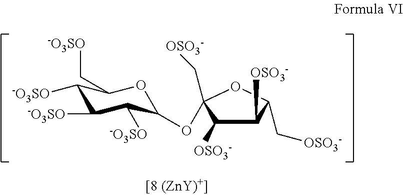

In this embodiment of the invention, the compound of the formula II is a compound of the general formula I wherein n is 0.

The present invention also provides a process for preparing these compounds.

The compounds of the general formula I are prepared according to scheme 1:

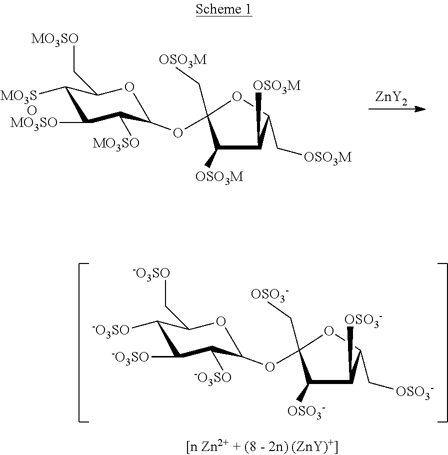

wherein:

M represents K, Na, or H

Y represents OH, Cl, Br, I, NO₃, BF₄, C₆H₅O₇, CH₃CO₂, CF₃CO₂, or —OCH₃

0≦n≦4

n is an integer.

The compounds of the general formula I are obtained from potassium sucrose octasulfate (M represents K), sodium sucrose octasulfate (M represents Na) or the acidic form of sucrose octasulfate (M represents H). In the case where the starting sucrose octasulfate salt is the sodium or potassium salt, a step of potassium or sodium ion exchange with protons is carried out by loading onto an ion exchange resin. The acidic form of sucrose octasulfate is added with either an inorganic zinc salt, selected from Zn(OH)₂, ZnCl₂, ZnBr₂, ZnI₂, Zn(NO₃)₂, or Zn(BF₄)₂, or an organic zinc salt, selected from Zn(CH₃CO₂)₂, Zn(CF₃CO₂)₂, Zn₃(C₆H₅O₇)₂, or Zn(CH₃O)₂. The zinc salt is added in amounts needed to yield a compound of the general formula I wherein the number n is an integer of between, or equal to, 0 and 4.

The invention thus covers the following preparation processes.

A process for preparing compounds of the general formula I comprising the following steps:

1) dissolving a sucrose octasulfate salt in water;

2) loading said salt onto an ion exchange column ;

3) adding a zinc salt;

4) precipitating zinc sucrose octasulfate.

In one embodiment of the invention, the sucrose octasulfate salt of step 1 is selected from potassium sucrose octasulfate or sodium sucrose octasulfate.

After passage through the ion exchange column, the sucrose octasulfate salt in acid form is obtained. More preferably, it is a cation exchange resin. In one embodiment of the invention, the cation exchange resin is Amberlite.

In one embodiment of the invention, the zinc salt is selected from either inorganic zinc salts such as Zn(OH)₂, ZnO, ZnCl₂, ZnBr₂, ZnI₂, Zn(NO₃)₂, or Zn(BF₄)₂, or organic zinc salts such as Zn(CH₃CO₂)₂, Zn(CF₃CO₂)₂, Zn₃(C₆H₅O₇)₂, or Zn(CH₃O)₂. The zinc salt is for example zinc hydroxide Zn(OH)₂.

In one embodiment of the invention, the precipitation of zinc sucrose octasulftate is carried out by adding acetone.

A process for preparing compounds of the general formula I comprising the following steps:

1) dissolving in water sucrose octasulfate in acid form;

2) adding a zinc salt;

3) precipitating zinc sucrose octasulfate.

The zinc salt is selected from either inorganic zinc salts such as Zn(OH)₂, ZnO, ZnCl₂, ZnBr₂, ZnI₂, Zn(NO₃)₂, or Zn(BF₄)₂, or organic zinc salts such as Zn(CH₃CO₂)₂, Zn(CF₃CO₂)₂, Zn₃(C₆H₅O₇)₂, or Zn(CH₃O)₂.

In one embodiment of the invention, the precipitation of the zinc sucrose octasulftate is carried out by adding acetone.

The compounds of the formula I according to the invention can be administered by topical or oral route. In particular the compound can be administered topically in a suitable formulation. The dose levels of the compounds of the formula I in the compositions of the invention can be adjusted to obtain an amount of active ingredient which is effective to achieve the desired therapeutic and/or cosmetic response for a composition suited for the dosing method. The selected dose level is thus dependent on the desired therapeutic and/or cosmetic effect, the route of administration, the desired duration of the treatment and other factors.

The invention therefore also relates to a pharmaceutical and/or cosmetic composition comprising at least one compound of the general formula I and a pharmaceutically and/or cosmetologically acceptable excipient.

The invention also relates to a medical device comprising at least one compound of the general formula I and a pharmaceutically or cosmetologically acceptable excipient.

The phrase <<pharmaceutically and/or cosmetologically acceptable>> refers to molecular entities and compositions which do not bring about any adverse side effects, allergic or other unwanted reaction when they are administered to animals or humans.

In one embodiment, the composition according to the invention contains zinc sucrose octasulfate according to the general formula I in an amount of between 0.01 and 30% by weight.

In one embodiment according to the invention, the composition comprises the compound of the formula II.

The pharmaceutically and/or cosmetologically acceptable excipient for obtaining a composition according to the invention is selected so as to be suited for topical or oral administration.

Advantageously, the topical form is selected from the group consisting of a milk, a cream, a balm, an oil, a lotion, a gel, of a foaming gel, an ointment, a spray, a paste, a patch, a suppository, etc.

Advantageously, the oral form is selected from the group consisting of a gum, a lozenge, a tablet, a cooked sugar, a drinking gel, a powder for dissolution, etc.

For the purpose of the present invention, the topical form includes topical dosage forms for use on skin, for oral use (oral mucosa), for genital use (anal, vaginal mucosa) and/or for gastric use.

For the purpose of the present invention, the oral form includes oral dosage forms for oral use (oral mucosa) and/or for gastric use.

The pharmaceutical and/or cosmetic compositions according to the present invention are designed for enhancing wound healing.

The antimicrobial properties of zinc have been well described. The pharmaceutical and/or cosmetic compositions according to the present invention therefore further intend to provide protection against microbial infections.

Thus, the pharmaceutical and/or cosmetic compositions according to the present invention enhance wound healing and/or provide protection against microbial infections.

In vitro studies showed that the compounds according to the invention induce keratinocyte migration. In comparison potassium sucrose octasulfate and sodium sucrose octasulfate do not induce cell migration.

Thus, surprisingly, unlike both potassium sucrose octasulfate and sodium sucrose octasulfate, zinc sucrose octasulfate has been found to display highly valuable properties on keratinocyte migration and therefore to be suitable for skin healing.

The compounds of the invention can thus be used for the treatment of the skin particularly in the healing process and for enhancing its esthetic appearance.

The compounds of the invention can thus be used for the preparation of compositions and pharmaceutical and/or cosmetic products to enhance wound healing.

Thus another object of the present invention relates to a compound according to the present invention for use as a medicament.

Another object of the present invention also relates to a compound according to the present invention for use as a cosmetic active principle.

Another object of the present invention further relates to a compound according to the present invention for use as a medicament and/or cosmetic active principle.

In one particular embodiment of the invention, the compounds of the formula I are used for the treatment of skin.

More particularly, the compounds of the formula I are used to enhance wound healing. More particularly, the invention relates to wound healing of acute wounds such as for example grazes, burns, radiation dermatitis, or chronic wounds such as for example ulcers, bed sores, and diabetic foot.

More particularly, the invention relates to wound healing of burns (of thermal, mechanical, chemical, radiation origin), radiation dermatitis, various rashes, dermatitis, grazes, scratches, scrapes, cuts, leg ulcers, bed sores, diabetic wounds, stomach ulcers, mouth sores, various wounds in the oral environment, scar acne, cryotherapy scars, post-surgery or post-dermatology plastic surgery scars (laser, hair removing, peeling, injection), blisters, cheilitis, eczema, diaper rash, dermatoporosis, etc.

In one particular embodiment of the invention, the compounds of the formula I are used to enhance wound healing and/or to protect against microbial infections.

The following examples are illustrative and not limiting.

### EXAMPLE 1

### Preparation of Compound of the Formula II

a. Preoaration Procedure

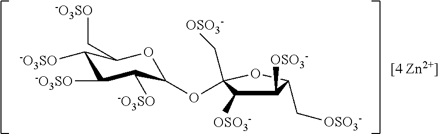

A 100-mL round bottom flask is charged with a solution of potassium sucrose octasulfate (1.50 g; 1.16 mmol, 1.00 equiv, 99%) in water (20 ml). The solution is loaded onto a column (Φ40×500 mm) comprising 250 g of ion exchange resin Amberlite IR 120 H at a flow rate of 2-3 mL/min at 0° C. The acidic fraction (120 mL; pH=1.2) is collected and immediately neutralized by adding freshly prepared zinc hydroxide (600 mg; 5.76 mmol; 5.05 equiv, 95%). The resulting mixture, which is hazy at about pH 6, is left under stirring overnight (about 12 hrs) at room temperature. These steps were all carried out in the dark by wrapping the reaction medium with aluminum foil.

The mixture is then filtered and 360 mL of acetone are added to the filtrate. The resulting mixture is allowed to stand overnight. The supernatant is decanted and the remaining syrup is washed with acetone. Zinc sucrose octasulfate is obtained as a white solid (0.40 g; 28%).

¹H NMR (D₂O, ppm): δ: 4.02-4.50 (m, 10 H); 4.53-4.71 (m, 2 H); 5.03 (d, J=8.1 Hz, 1 H); 5.70-5.71 (d, J=3.6 Hz, 1 H).

b. Determination of Sucrose Octasulfate

The amount of sucrose octasulfate is determined by a spectrophotometric assay with anthrone (Brooks, J.; Griffin, V. K.; Kaftan, M. W.; <<A Modified Method for Total Carbohydrate Analysis of Glucose Syrups, Maltodextrins, and Other Starch Hydrolysis Products>>; Cereal Chemistry, 63, 5, 465-466, 1986).

A standard solution of anthrone is prepared by dissolving 50 mg of anthrone in a mixture of 10 mL of distilled water and 90 mL of concentrated sulfuric acid.

The zinc sucrose octasulfate is dehydrated beforehand in vacuo (about 23.5 Pa) at 30° C. for 6 hrs. Three samples having a volume of 0.3, 0.6 and 0.7 mL respectively, of an aqueous solution of zinc sucrose octasulfate (0.4018 mg/mL) are diluted to 2 ml with distilled water. 6.0 mL of the standard solution of anthrone are added to each solution. The solutions obtained are heated in a water-bath for 10 min. After immediate cooling to room temperature, the absorbance of each solution is measured at 620 nm, using sucrose as a reference (the results are shown in Table 1).

TABLE 1
Determination of sucrose with anthrone

| Sample | | Concentration of | Concentration of |
|---|---|---|---|
| volume | | diluted sample | sucrose octasulfate |
| (mL) | Absorbance | (mM)^{a} | (mM)^{b} |
| 0.3 | 0.167 | 0.0427 | 0.285 |
| 0.6 | 0.326 | 0.0834 | 0.278 |
| 0.7 | 0.380 | 0.0927 | 0.324 |

^{a}diluted to 2 mL with distilled water
^{b}average concentration: 0.296 mM

The amount of sucrose octasulfate in the zinc sucrose octasulfate is: 0.296 mM/0.4018 mg/mL=0.737 mmol/g.

c. Determination of Zinc Content

The zinc content was determined by titration with EDTA.

The zinc sucrose octasulfate (0.2009 g) is dissolved in deionized water (250 mL). The salt is titrated with an aqueous solution of EDTA (0.0101 M) containing 6 mL of hexamethylene tetramine (20%) as a buffer solution and 2 drops of xylenol orange (0.2%) as a color indicator (Table 2).

TABLE 2
Determination of zinc content with EDTA

| | 1 | 2 | 3 | average |
|---|---|---|---|---|
| V_{Zn2+SOS-Zn} (mL) | 25.00 | 25.00 | 25.00 | 25.00 |
| V_{EDTA} (mL) | 5.67 | 5.70 | 5.72 | 5.70 |
| C_{Zn2+SOS-Zn} (mM) | 2.29 | 2.30 | 2.31 | 2.30 |

The amount of zinc in the zinc sucrose octasulfate is: 2.30 mM×0.250 l/0.2009 g=2.86 mmol/g.

The zinc/sucrose octasulfate ratio is 2.86/0.737, that is 3.88.

d. Analysis for Potassium Impurities

UFLC (Ultra Fast Liquid Chromatography) was used to detect the presence of any potassium from the starting material.

Conditions:

Column: Merck SeQuant ZIC_HILIC 150×4.6 mm 5 μm 200 A

Mobile Phase A: Acetonitrile

Mobile Phase B: 100 mM Ammonium Acetate pH=5.0

Flow rate: 0.6 ml/min

Column Temperature: 35° C.

Detector: ELSD

Gradient: 15% to 85% B over 11.0 minutes, 85% to 95% B over 8.0 minutes, 95% B for 5 minutes

Results:

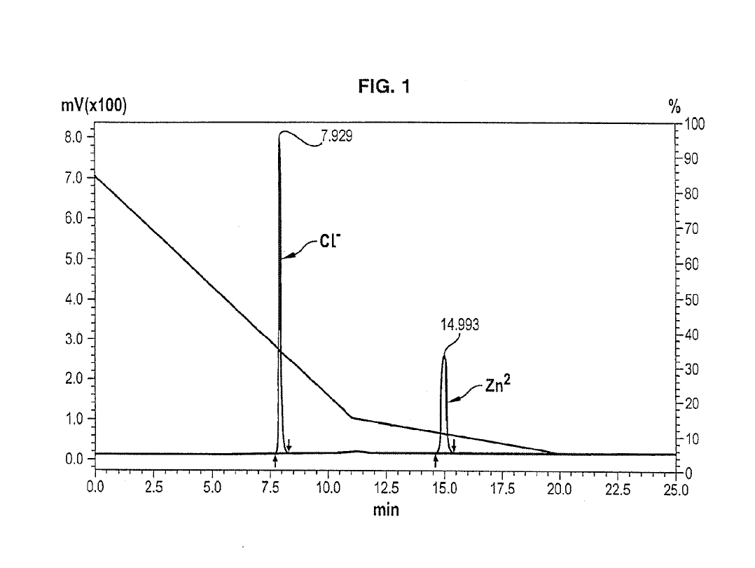

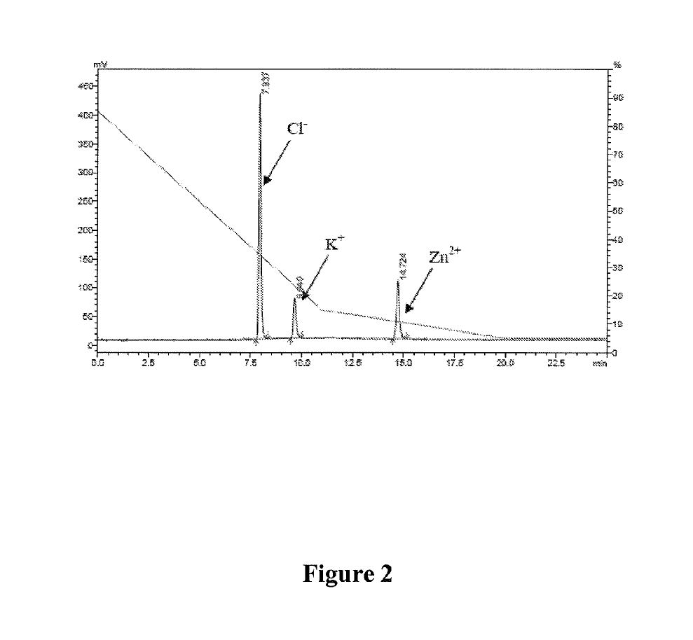

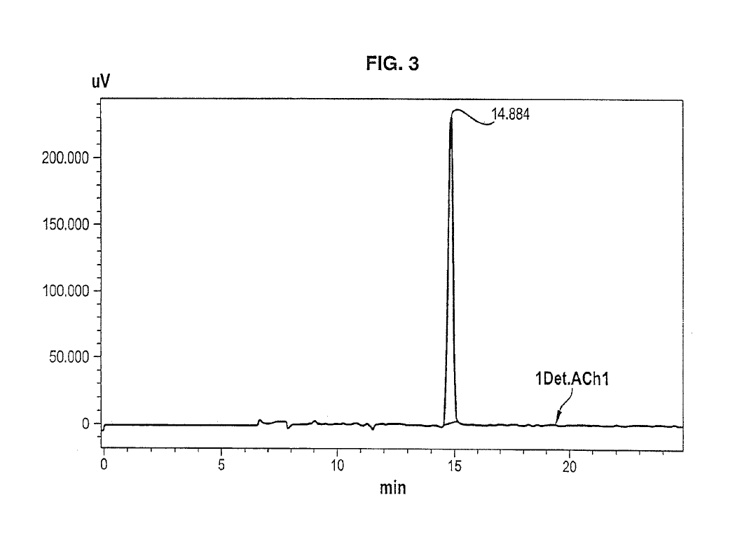

The zinc sucrose octasulfate obtained according to Example 1 and two controls were analyzed by UFLC:
ZnCl₂ (see FIG. 1)
ZnCl₂+KCl (see FIG. 2)
zinc sucrose octasulfate (Formula II) (see FIG. 3)

The analysis confirmed the absence of potassium in the synthesized product. Zinc only is present as a cation.

### EXAMPLE 2

### In Vitro Cell Migration Assay

Epithelial cell migration is a substantial stage of the development and processes of tissue repair, such as embryogenesis and wound healing.

The mechanisms of initiation, coordination and termination of movement of the cells are not fully understood, however, the essential role of cell migration is well established (Santoro M. M. and Gaudino G. Cellular and molecular facets of keratinocyte reepithelization during wound healing Exp. Cell. Res. 304 (1): 274-286, 2005; Werner S. and Grose R. Regulation of wound healing by growth factors and cytokines Physiol. Rev. 3(3): 835-870, 2003; Steffensen B, Akkinen L., Lariava H. Proteolytic events of wound healing-coordinated interactions among matrix metalloproteinases (MMPs), integrins, and extracellular matrix molecules Crit. Rev. Oral Biol. Med. 12(5): 373-398, 2001).

During skin healing and in dermatologic chronic inflammatory conditions, the keratinocytes are “activated” to start the migration processes. The cells have then their phenotype controlled on the one hand by interactions with the extracellular matrix and on the other hand by cell-cell interactions (McMillan J R, Akiyama M., Shimizu H. Epidermal basement membrane zone component: ultrastructural distribution and molecular interactions J. Derm. Sc. 31: 169-177, 2003). Keratinocytes of the basal seat of the borders of a wound migrate over and cover the wound.

In fact, keratinocytes are activated when they come into contact with fibronectin, interstitial dermal collagen (type 1), collagen IV, and laminin 5 from the basal lamina. They are also controlled by some polypeptide growth factors such as TGFβ, TGFα and EGF. Moreover, cytokines (IL1, TNFα) and chemokines (RANTES and IL-8) also help increasing the rate of wound re-epithelization, upon keratinocyte activation (Szabo I., Wetzel M. A., Rogers T J. Cell-Density-Regulated Chemotactic Responsiveness of Keratinocytes In Vitro J. Invest. Dermatol. 117: 1083-1090, 2001).

Purpose of the Study

The purpose of this study was to assess the effect of zinc sucrose octasulfate on cell migration of keratinocyte cell lines HaCAT, using an Oris Cell Migration Assay Kit (Platypus Technologies). This study was carried out in comparison with potassium sucrose octasulfate, and sodium sucrose octasulfate.

Thus, the 3 sucroses were analyzed for their effect on migration of HaCat cells.

Materials and Methods

a. Biologic Material

Spontaneously immortalized human keratinocyte cell line HaCaT, frequently referred to in literature as a standard model.

b. Cell Migration Protocol

The protocol used for the cell migration study is based on the use of a 96-well Oris Cell Migration Assay kit (Platypus Technologies-TEBU), providing miniaturization and quantification of this cell process. It is designated by code number QRD/TO/154/107.

The principle of this assay is to investigate the cell migration towards the centre of the well of a 96-well plate. A stopper is placed in some wells, in order to create a detection zone of 2 mm in diameter. Then the stoppers are removed after the cells have well adhered to the surface around them, thus allowing the cells to migrate towards the detection zone. The plates without the stoppers and with the active substances are incubated at 37° C. for 24 hours in DMEM 0% SVF. Following this, the amount of cells located in the zone where the stopper was, is analyzed, in order to assess cell migration. A mask restricts visualization and reading to cells located in this zone only. For each condition, the average for 4 to 8 wells is calculated.

c. Test Products

Positive control: TGFβ1

Potassium sucrose octasulfate (SOS-K)

Sodium sucrose octasulfate (SOS-Na)

Zinc sucrose octasulfate according to Example 1 (SOS-Zn)

d. Analysis of the Results

The results are expressed as OD (proportional to the amount of migrated cells).

Percent activity with regard to the negative control is calculated as:

$$\frac{OD treated}{OD negative control} \times 100$$

Percent activity with regard to TGFβ is calculated as:

$$\frac{OD treated - OD negative control}{OD TGF β - OD negative control} \times 100$$

Results

The 3 sucroses at various concentrations were analyzed for their effect on HaCat cell migration, in duplicate (Tables 3 and 4).

a. Experiment 1

TABLE 3
Results of Experiment 1 - Effect of the
3 sucroses on keratinocyte migration

| | SOS-Zn | SOS-Na | SOS-K | TGFβ | Negative |
|---|---|---|---|---|---|
| | 1 μM | 1 μM | 1 μM | 5 ng/mL | control |
| Average OD | 306.9 | 28.6 | 99.5 | 258.6 | 87.7 |
| % activity/ | 350.0 | 32.6 | 113.5 | 294.9 | |
| negative | | | | | |
| control | | | | | |
| % activity/ | 128.3 | −34.6 | 6.9 | | |
| TGFβ | | | | | |

b. Experiment 2

TABLE 4
Results of Experiment 2 - Effect of the
3 sucroses on keratinocyte migration

| | SOS-Zn | SOS-Na | SOS-K | TGFβ | Negative |
|---|---|---|---|---|---|
| | 1 μM | 1 μM | 1 μM | 5 ng/mL | control |
| Average OD | 417.8 | 142.3 | 321.3 | 436.3 | 237.4 |
| % activity/ | 176.0 | 59.9 | 135.3 | 183.8 | |
| negative | | | | | |
| control | | | | | |
| % activity/ | 90.7 | −47.1 | 42.2 | | |
| TGFβ | | | | | |

It was found that:
TGFβ at 5 ng/mL, the positive control for the experiments, induces keratinocyte migration reproducibly;
zinc sucrose octasulfate (SOS-Zn) also induces cell migration reproducibly at 1 μM. At such concentration it is as active as the positive control TGFβ;
at the concentrations tested, potassium sucrose octasulfate (SOS-K) and sodium sucrose octasulfate (SOS-Na) do not induce keratinocyte migration.

Using this cell migration kit, the effect of the 3 sucroses on keratinocyte migration was studied.

We showed that zinc sucrose octasulfate induces keratinocyte migration. In comparison, potassium sucrose octasulfate and sodium sucrose octasulfate do not induce cell migration.

Unlike potassium sucrose octasulfate and sodium sucrose octasulfate, zinc sucrose octasulfate has been found to have highly valuable properties on the keratinocyte migration and therefore to be suitable for skin healing.

1. A compound of the general formula I 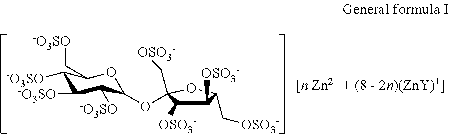
wherein:
0≦n≦4
n is an integer
Y represents OH, Cl, Br, I, NO₃, BF₄, C₆H₅O₇, CH₃CO₂, CF₃CO₂, or —OCH₃.

2. The compound according to claim 1 characterized in that it is a compound of the formula II

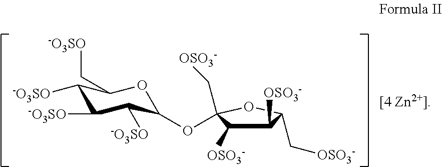

3. A process for preparing compounds of the general formula I according to claim 1 characterized in that it comprises the following steps:
1) dissolving in water a sucrose octasulfate salt;
2) loading said salt onto an ion exchange column;
3) adding a zinc salt;
4) precipitating zinc sucrose octasulfate.

4. The process according to claim 3 characterized in that the sucrose octasulfate salt is potassium sucrose octasulfate or sodium sucrose octasulfate.

5. A process for preparing compounds of the general formula I characterized in that it comprises the following steps:
1) dissolving in water sucrose octasulfate in acid form;
2) adding a zinc salt;
3) precipitating zinc sucrose octasulfate.

6. The process according to any of claims 3 to 5, characterized in that said zinc salt is an inorganic zinc salt, selected from Zn(OH)₂, ZnCl₂, ZnBr₂, ZnIr, Zn(NO₃)₂, or Zn(BF₄)₂, or an organic zinc salt, selected from Zn(CH₃CO₂)₂, Zn(CF₃CO₂)₂, Zn₃(C₆H₅O₇)₂, or Zn(CH₃O)₂.

7. A pharmaceutical and/or cosmetic composition comprising at least one compound according to any of claims 1 to 2 and a pharmaceutically and/or cosmetologically acceptable excipient.

8. The compound according to any of claims 1 to 2, for use as a medicament.

9. The compound according to claim 8, for the treatment of skin or mucosa.

10. The compound according to claim 9, for enhancing wound healing.

11. The compound according to claim 10 characterized in that it is suitable for healing burns and acute or chronic wounds.

12. The compound according to claim 10 characterized in that it is suitable for healing burns, radiation dermatitis, various rashes, dermatitis, grazes, scratches, scrapes, cuts, leg ulcers, bed sores, diabetic wounds, stomach ulcers, mouth sores, various wounds in the oral environment, scar acne, cryotherapy scars, post-surgery or post-dermatology plastic surgery scars, blisters, cheilitis, eczema, diaper rash, and dermatoporosis.

13. A process of cosmetic treatment of skin and mucosa, characterized in that it involves the application as an active principle of at least one compound according to either of claims 1 and 2.
